(19) United States
(12) Patent Application Publication
(10) Pub. No.: US 2022/0087706 A1
(43) Pub. Date: Mar. 24, 2022

(54) ULTRASOUND TREATMENT TOOL, ULTRASOUND TREATMENT SYSTEM, AND ENDOSCOPIC SURGERY SYSTEM

(71) Applicant: OLYMPUS CORPORATION, Tokyo (JP)

(72) Inventors: Yasuyuki MATSUMURA, Hachioji-shi (JP); Takamitsu SAKAMOTO, Hachioji-shi (JP); Ken FUJISAKI, Sagamihara-shi (JP); Naoyuki SUIDO, Sagamihara-shi (JP)

(73) Assignee: OLYMPUS CORPORATION, Tokyo (JP)

(21) Appl. No.: 17/538,118

(22) Filed: Nov. 30, 2021

Related U.S. Application Data
(63) Continuation of application No. PCT/JP2019/023227, filed on Jun. 12, 2019.

(51) Int. Cl. A61B 17/32; A61B 1/317; A61B 1/015

(52) U.S. Cl. CPC A61B 17/320068; A61B 1/317; A61B 2017/32009; A61B 2017/32007; A61B 2017/320084; A61B 1/015

(57) ABSTRACT

An ultrasound treatment tool that includes: a probe that includes a main body and a treating portion; and a first tubular portion. The first tubular portion includes at least one through hole that is formed in a wall of the first tubular portion so as to be in fluid communication with a liquid channel in the first tubular portion, and an opening that is formed at a distal end of the first tubular portion. A total opening area of the through hole(s) is larger than a cross-sectional opening area of the liquid channel at the opening.

### CROSS-REFERENCE TO RELATED APPLICATION

This application is a continuation of International Application No. PCT/JP2019/023227, filed on Jun. 12, 2019, the entire contents of which are incorporated herein by reference.

### BACKGROUND

### 1. Technical Field

The present disclosure relates to an ultrasound treatment tool, an ultrasound treatment system, and an endoscopic surgery system.

### 2. Related Art

In arthroscopic surgery, which is endoscopic surgery in orthopedics, it has been known to treat a portion to be treated by using an ultrasound treatment tool while providing a field of arthroscope by injecting a perfusion solution such as a physiological saline solution to expand an articular cavity.

### SUMMARY

In some embodiments, an ultrasound treatment tool includes: a probe that includes a main body and a treating portion, and a first tubular portion that is formed in a tubular shape with the probe inserted therein. The first tubular portion includes at least one through hole that is formed in a wall of the first tubular portion so as to be in fluid communication with a liquid channel of liquid of the first tubular portion, and an opening that is formed at a distal end of the first tubular portion. A total opening area of all of the through hole(s) is larger than a cross-sectional opening area of the liquid channel at the opening.

In some embodiments, an ultrasound treatment tool includes: a probe that includes a main body extending along a central axis and a treating portion disposed at a distal end of the main body, and a first tubular portion that is formed in a tubular shape with the probe inserted therein. The first tubular portion includes at least one through hole that is formed in a wall of the first tubular portion so as to be in fluid communication with a liquid channel of the first tubular portion, and an opening that is formed at a distal end of the first tubular portion. An antinode position of a standing wave of the ultrasonic vibration is positioned at the treating portion, and the at least one through hole is formed at a position along the central axis that corresponds to a node position nearest to the antinode position, or formed on a distal end side relative to the node position nearest to the antinode position.

In some embodiments, an ultrasound treatment system includes: the ultrasound treatment tool; and a driving device configured to drive the ultrasound treatment tool.

In some embodiments, an endoscopic surgery system includes: the ultrasound treatment system; an endoscope configured to acquire image data of the target site in an articular cavity; and a perfusion device configured to perfuse a liquid into the articular cavity.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an entire configuration of an endoscopic surgery system according to an exemplary embodiment;

FIG. 2 is a diagram illustrating an entire configuration of an ultrasound treatment system according to an exemplary embodiment;

FIG. 3 is a cross-section of a portion around a distal end portion of an ultrasound treatment tool;

FIG. 4 is a diagram illustrating an antinode position of vibration and a node position of vibration when ultrasonic vibration is input to a probe of the ultrasound treatment tool;

FIG. 5 is a diagram illustrating a flow of a perfusion solution that is caused by causing the probe to make ultrasonic vibrations;

FIG. 6A is a diagram illustrating a field of view of an arthroscope showing a state in which fragments of a living tissue are dispersed in the perfusion solution;

FIG. 6B is a diagram illustrating a state in which fragments of a living tissue dispersed in the perfusion solution are sucked in from a suction port of an outer sheath;

FIG. 7 is a cross-section taken along a ling A1-A1 in FIG. 3;

FIG. 8A is a diagram illustrating a step of making a portal in a skin near a joint;

FIG. 8B is a diagram illustrating a step of inserting the arthroscope and the ultrasound treatment tool in the portal;

FIG. 8C is a diagram illustrating a field of view of the arthroscope showing a step of positioning of a treating portion with respect to a part to be treated, and of confirming that a drain hole of the outer sheath is not present in the field of view of the arthroscope;

FIG. 8D is a diagram illustrating a field of view of the arthroscope to show a step of driving the ultrasound treatment tool;

FIG. 9A is a side view of a first example of the treating portion of the ultrasound treatment tool;

FIG. 9B is a front view of the first example of the treating portion of the ultrasound treatment tool;

FIG. 10 is a perspective view of a second example of the treating portion of the ultrasound treatment tool;

FIG. 11A is a diagram illustrating an example of a shape of the drain hole to be formed in the outer sheath;

FIG. 11B is a diagram illustrating an example of a shape of the drain hole to be formed in the outer sheath;

FIG. 11C is a diagram illustrating an example of a shape of the drain hole to be formed in the outer sheath;

FIG. 11D is a diagram illustrating an example of a shape of the drain hole to be formed in the outer sheath;

FIG. 12 is a cross-section around a distal end portion of the ultrasound treatment tool in which a through hole is formed in a probe;

FIG. 13 is a diagram illustrating an entire configuration of an ultrasound treatment system that is included in an endoscopic surgery system according to an exemplary embodiment;

FIG. 14 is a diagram illustrating a flow of a perfusion solution that is caused by causing a probe to make ultrasonic vibrations;

FIG. 15 is a diagram of the probe and a probe cover when viewed along a longitudinal direction from a distal end side of the probe to a proximal end side;

FIG. 16 is a cross-section taken along a line A2-A2 in FIG. 14; and

FIG. 17 is a diagram illustrating an entire configuration of an ultrasound treatment system that is included in an endoscopic surgery system according to an exemplary embodiment.

### DETAILED DESCRIPTION

Hereinafter, an exemplary embodiment of an ultrasound treatment tool, an ultrasound treatment system, an endoscopic surgery system, and an endoscopic surgery method according to the disclosure will be explained. The embodiment is not intended to limit the disclosure.

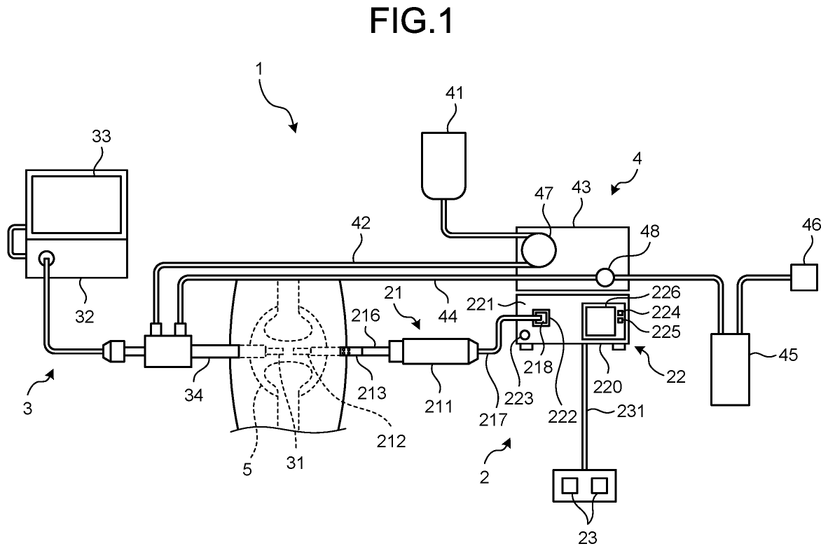

FIG. 1 is a diagram illustrating an entire configuration of an endoscopic surgery system 1 according to the present embodiment. As illustrated in FIG. 1, the endoscopic surgery system 1 according to the present embodiment includes an ultrasound treatment system 2, an arthroscopic device 3, and a perfusion device 4.

The arthroscopic device 3 includes an arthroscope 31 that is an endoscope to acquire image data of a part to be treated in an articular cavity 5 of a patient, an image processing device 32 that converts a video of the arthroscope 31 into an electrical signal, a monitor 33 that shows a video based on the electrical signal of the image processing device 32, and a cannula 34. The arthroscope 31 is inserted into the articular cavity 5 through the cannula 34 punctured into the articular cavity 5 of the patient.

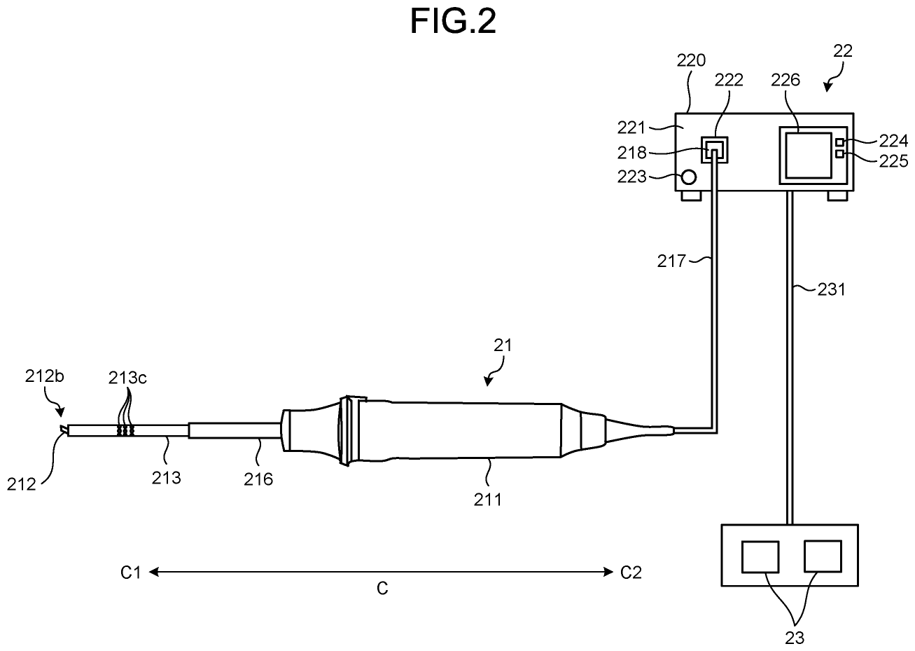

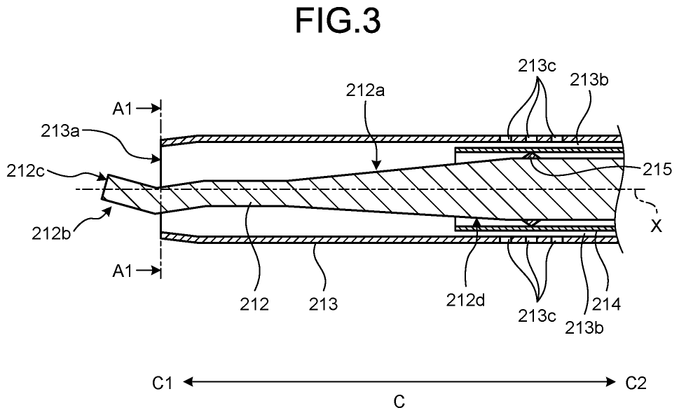

FIG. 2 is a diagram illustrating an entire configuration of the ultrasound treatment system 2 according to the present embodiment. The ultrasound treatment system 2 includes an ultrasound treatment tool 21, a driving device 22, and a foot switch 23. FIG. 3 is a cross-section of a portion around a distal end portion of the ultrasound treatment tool 21.

The ultrasound treatment tool 21 includes a treatment-tool main body 211, a rod-shaped probe 212, an outer sheath 213 that is a first tubular portion to protect the probe 212 by covering the probe 212, an inner sheath 214 that is a second tubular portion provided inside the outer sheath 213, a seal ring 215, a resin tube 216, a cable 217, and a connector 218. In the present embodiment, a direction parallel to an axis X (refer to FIG. 3) of the probe 212 is denoted as longitudinal direction C. Moreover, in the present embodiment, one of two directions parallel to the longitudinal direction C of the probe 212 is denoted as distal end direction C1, and a direction opposite to the distal end direction C1 is denoted as proximal end direction C2.

The treatment-tool main body 211 has a tubular shape, and houses an ultrasound transducer that is constituted of a piezoelectric body using lead zirconate titanate (PZT) and the like, a driving circuit to drive the ultrasound transducer, and the like thereinside.

The probe 212 is formed in a rod shape with a metallic material having biocompatibility, such as titanium alloy. The probe 212 includes a probe main body 212a that extends in a rod shape, and a treating portion 212b in a rectangular shape that is arranged on a side in the distal end direction C1 of the probe main body 212a. A proximal end portion of the probe main body 212a is connected to the treatment-tool main body 211. At a distal end of the treating portion 212b, a cutting area 212c to be brought into contact with a living tissue is arranged.

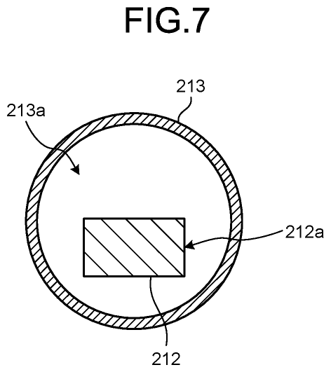

The probe main body 212a is formed to have a cross-section in a direction perpendicular to the longitudinal direction C in a rectangular shape (refer to FIG. 7), and has a tapered portion 212d that becomes thinner from the proximal end direction C2 side toward the distal end direction C1 side in the longitudinal direction C. The distal end direction C1 side of the probe main body 212a is bend toward a first direction that is different from the direction along the longitudinal direction C. Moreover, the treating portion 212b is bent toward a second direction that is different from the longitudinal direction C and from the first direction, relative to the probe main body 212a.

The outer sheath 213 is in a tubular shape thinner than the treatment-tool main body 211, and covers a part of an outer circumferential surface of the probe 212 from the treatment-tool main body 211 to a portion near a boundary between the probe main body 212a and the treating portion 212b in the probe 212. At a distal end of the outer sheath 213, a suction port 213a, which is an opening, is formed. Space is formed between an inner circumferential surface of the outer sheath 213 and an outer circumferential surface of the inner sheath 214, to serve as a suction channel 213b through which the perfusion solution can flow through. Moreover, in a wall portion of the outer sheath 213, plural drain holes 213c that are through holes communicating between the outer circumferential surface and the inner circumferential surface of the outer sheath 213 are formed. The drain holes 213c respectively communicate with the suction channel 213b. Furthermore, on the outer circumferential surface of the outer sheath 213 on the proximal end direction C2 side, a resin tube 216 is arranged.

A first end side of the cable 217 is electrically connected to the driving circuit and the like inside the treatment-tool main body 211. To a second end side of the cable 217, the connecter 218 is connected.

The driving device 22 includes plural electronic parts and the like constituting an electronic circuit and the like in a casing 220. In a front panel 221 of the driving device 22, a connector 222, a power switch 223, two operating switches 224, 225, a monitor 226, and the like are arranged. To the connector 222, the connector 218 of the cable 217 in the ultrasound treatment tool 21 is detachably connected. For the ultrasound treatment tool 21 and the driving device 22, by connecting the connector 218 and the connector 222, supply of a driving power, communication of a control signal, and the like are performed through the cable 217.

The foot switch 23 is connected to the driving device 22 by a cable 231. In the ultrasound treatment system 2, as a surgeon operates the foot switch 23, ultrasonic vibrations are generated by the ultrasound transducer of the ultrasound treatment tool 21 by a driving power from the driving device 22. Moreover, setting of an output level of ultrasonic vibration is performed by the surgeon operating the operating switches 224, 225 of the driving device 22.

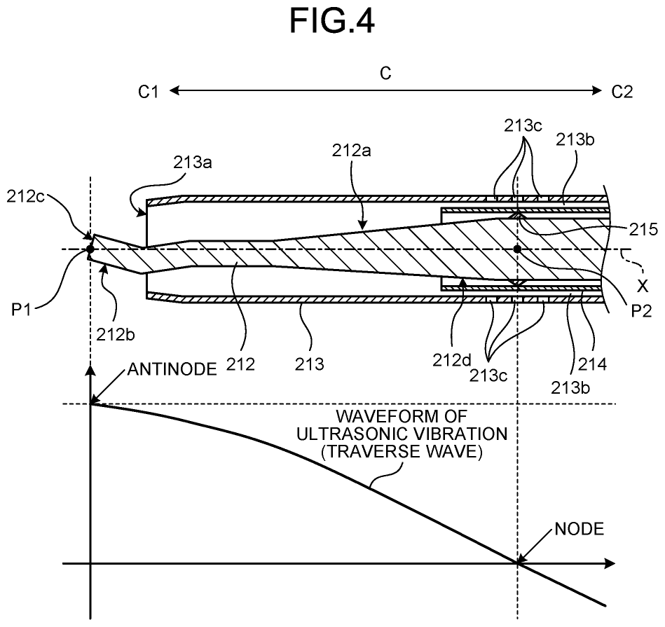

FIG. 4 is a diagram illustrating an antinode position P1 of vibration and a node position P2 of vibration when ultrasonic vibration is input to the probe 212 of the ultrasound treatment tool 21.

The probe 212 has a length that the antinode position P1 of vibration is defined for the treating portion 212b when ultrasonic vibration is input to the proximal end portion of the probe main body 212a from the ultrasound transducer of the treatment-tool main body 211, and the ultrasound vibration is transmitted to the treating portion 212b from the probe main body 212a. Moreover, the antinode position P1 of vibration is positioned at the cutting area 212c to be in contact with a living tissue in the treating portion 212b.

Furthermore, when ultrasound vibration is transmitted to the probe 212, the node position P2 of the first vibration on the proximal end direction C2 side relative to the antinode portion P1 of vibration is positioned at the probe main body 212a. The node position P2 of vibration varies according to a frequency of ultrasound vibration and, for example, a frequency of the ultrasound vibration is 47 kHz in the present embodiment.

At a position corresponding to the node position P2 of vibration in the longitudinal direction C of the probe main body 212a, a resin seal ring 215 to prevent liquid from flowing into the inside of the inner sheath 214 is arranged. Moreover, the drain holes 213c formed in the outer sheath 213 are positioned at a position corresponding to the node position P2 of vibration or at a position close thereto, in the longitudinal direction C of the outer sheath 213.

That is, in the present embodiment, the antinode position P1 of a standing wave of ultrasound vibration is positioned near the distal end of the treating portion 212b, and at least one of the drain holes 213c is formed in the outer sheath 213 at a position corresponding to the node position P2 next to the antinode position P1.

Moreover, it is preferable that at least one of the drain holes 213c is formed in the outer sheath 213 at a position corresponding to the node position P2 of vibration, and that the rest of the drain holes 213c is formed in the outer sheath 213 at a position facing the tapered portion 212d in the probe main body 212a of the probe 212 and is formed in the outer sheath 213 on the distal end direction C1 side relative to the position corresponding to the node position P2 of vibration. Thus, the perfusion solution flowing toward the node position P2 of vibration from the suction port 213a of the outer sheath 213 can be drained efficiently from the drain holes 213c.

All of the drain holes 213c may be formed at a position facing the tapered portion 212d in the probe main body 212a of the probe 212 and is formed on the distal end direction C1 side relative to the position corresponding to the node position P2 of vibration. In this case, the drain holes 213c are preferable to be formed near an end on the proximal end direction C2 side of the tapered portion 212d. Thus, the perfusion solution can be efficiently drained from the drain holes 213c at a position at which the channel is narrowed by the tapered portion 212d, and the flow speed of perfusion solution is increased.

The perfusion device 4 includes a liquid container 41 that contains the perfusion solution, such as physiological saline solution, a liquid feeding tube 42, a first end of which is connected to the liquid container 41, a perfusion pump unit 43, a drainage tube 44, a drain container 45 to which a first end of the drainage tube 44 is connected, and a suction pump 46 that is connected to the drain container 45. A second end of a liquid feeding tube 42 is connected to the cannula 34, and the perfusion solution is fed inside the articular cavity 5 through a feeding channel formed in the cannula 34 from the liquid feeding tube 42.

Furthermore, a second end of the drainage tube 44 is connected to the cannula 34, and the perfusion solution is drained to the drainage tube 44 from the inside of the articular cavity 5 through the drainage channel formed in the cannula 34.

In the perfusion pump unit 43, a liquid feeding pump 47 is provided. To the liquid feeding pump 47, the liquid feeding tube 42 is connected, and by driving the liquid feeding pump 47, the perfusion solution is fed out to the liquid feeding tube 42 from the liquid container 41. Moreover, a drain valve 48 is arranged in the perfusion pump unit 43. To the drain valve 48, the drainage tube 44 is connected, and by opening and closing the drain valve 48, a flow of the perfusion solution in the drainage tube 44 to the drain container 45 relative to the drain valve 48 can be controlled. For example, when a surgeon starts treatment of a part to be treated 51 in the articular cavity 5 by using the endoscopic surgery system 1, by closing the drain valve 48, the inside of the articular cavity 5 is brought into an expanded state at a certain pressure with the perfusion solution so that favorable observation by the arthroscope 31 is possible. Moreover, when the perfusion solution inside the articular cavity 5 is exchanged, or the like, by opening the drain valve 48, the perfusion solution can be drained from the inside of the articular cavity 5.

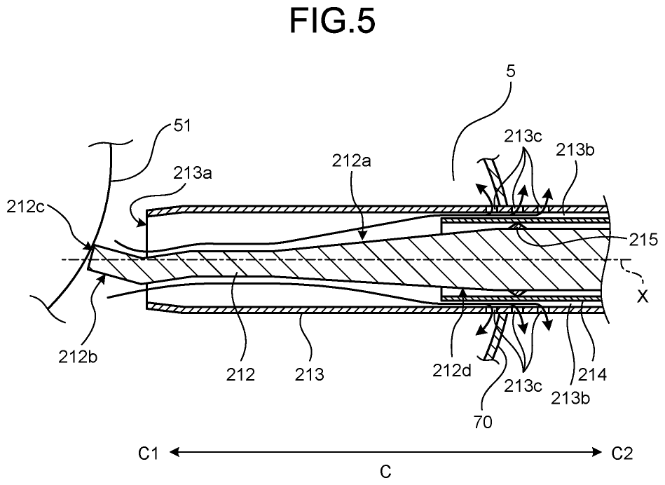

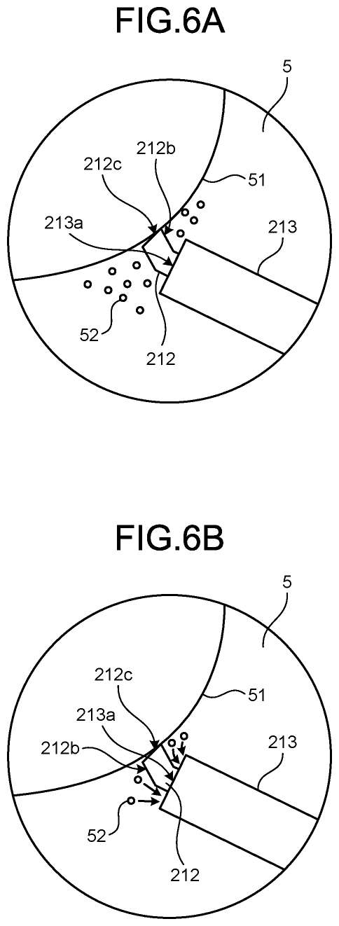

FIG. 5 is a diagram illustrating a flow of the perfusion solution that is caused by causing the probe 212 to make ultrasonic vibrations. Reference numeral 70 in FIG. 5 is a skin near a joint in which the part to be treated 51 is present. FIG. 6A is a diagram illustrating a field of view of the arthroscope showing a state in which fragments 52 of a living tissue are dispersed in the perfusion solution. FIG. 6B is a diagram illustrating a field of view of the arthroscope showing a state in which the fragments 52 of a living tissue dispersed in the perfusion solution are sucked in from the suction port 213a of the outer sheath 213.

As illustrated in FIG. 5, in the ultrasound treatment tool 21 according to the present embodiment, by the probe 212 making ultrasonic vibration in the longitudinal direction C, a flow of the perfusion solution to be drawn to the node position P2 of vibration from the suction port 213a of the outer sheath 213 is generated along the longitudinal direction C of the probe 212. Therefore, in the ultrasound treatment tool 21 according to the present embodiment, as illustrated in FIG. 6A, treatment is performed, bringing the cutting area 212c in the treating portion 212b of the probe 212 into contact with the part to be treated 51, and the perfusion solution including the fragments 52 of a living tissue, such as crushed bone fragments, generated by this treatment flows into the outer sheath 213 from the suction port 213a of the outer sheath 213 as illustrated in FIG. 6B. The perfusion solution that has flowed into the outer sheath 213 flows toward the proximal end direction C2 side in the outer sheath 213, passes through the suction channel 213b, and is drained from the drain holes 213c in the outer sheath 213. Thus, near the part to be treated 5, in other words, near a position at which the fragments 52 of a living tissue are generated by performing the treating portion 212b of the ultrasound treatment tool 21, the fragments 52 of a living tissue can be sucked and removed. Accordingly, even if it is difficult to insert a dedicated device to suck the fragments 52 of a living tissue generated by cutting in the articular cavity 5 in addition to the ultrasound treatment tool 21 and the arthroscope 31 because the inside of the articular cavity 5 is narrow, turbidity of the perfusion solution with the fragments 52 of a living tissue can be reduced near the part to be treated 51, and a clear visibility in a field of view of arthroscope can be obtained.

Generally, the thickness of a skin is 10 mm in maximum. Therefore, in the present embodiment, it is preferable to satisfy a relationship of thickness of the skin 70<size of the drain hole 213c, or thickness of the skin 70<range in which the drain holes 213c are formed. Thus, it is possible to suppress blocking of the drain holes 213c by the skin 70, and to drain the fragments 52 of a living tissue from the drain holes 213c.

Moreover, it is preferable that at least one of the drain holes 213c is formed in the outer sheath 213 at a position corresponding to the node position P2 of vibration, and that the rest of the drain holes 213c is formed in the outer sheath 213 at a position facing the tapered portion 212d in the probe main body 212a of the probe 212 and is formed in the outer sheath 213 on the distal end direction C1 side relative to a position corresponding to the node position P2 of vibration. Thus, the perfusion solution flowing toward the node position P2 of vibration from the suction port 213a of the outer sheath 213 can be efficiently drained from the drain holes 213c.

Furthermore, all of the drain holes 213c may be formed at a position facing the tapered portion 212d in the probe main body 212a of the probe 212 and is formed on the distal end direction C1 side relative to the position corresponding to the node position P2 of vibration. In this case, it is preferable that the drain holes 213c be formed near an end on the proximal end direction C2 side of the tapered portion 212d. Thus, at a position at which the flow channel is narrowed by the tapered portion 212d, and the flow speed of the perfusion solution becomes high, the perfusion solution can be efficiently drained from the drain holes 213c.

The size of each of the drain holes 213c formed in the outer sheath 213 is equal to or larger than an outer diameter of the fragment 52 of a living tissue in a size passable through the suction channel 213b. Moreover, a total sum of areas of the drain holes 213c formed in the outer sheath 213 is larger than an opening area of the suction port 213a of the outer sheath 213. This enables to make an amount of the perfusion solution drained from the drain holes 213c larger than an amount of the perfusion solution sucked from the suction port 213a of the outer sheath 213, and the perfusion solution can be efficiently drained from the inside of the outer sheath 213.

The opening area of the suction port 213a is an area acquired by subtracting an area of a cross-sectional area of the probe 212 from a cross-sectional area of the suction port 213a (area of a region surrounded by an inner circumferential surface of the outer sheath 213 on an A1-A1 cross-section) when the A1-A1 cross-section that is perpendicular to the axis of the probe 212 is viewed along the longitudinal direction from the distal end direction C1 side toward the proximal end direction C2 side of the probe 212 as illustrated in FIG. 7. In other words, the opening area of the suction port 213a is an area of a gap formed between the inner circumferential surface of the outer sheath 213 and the outer circumferential surface of the probe 212 when the A1-A1 cross-section is viewed along the longitudinal direction C from the distal end direction C1 side toward the proximal end direction C2 side of the probe 212.

Furthermore, in the ultrasound treatment tool 21 according to the present embodiment, because the perfusion solution sucked from the suction port 213a of the outer sheath 213 flows along the probe 212, a cooling performance of the probe 212 by the perfusion solution can be improved.

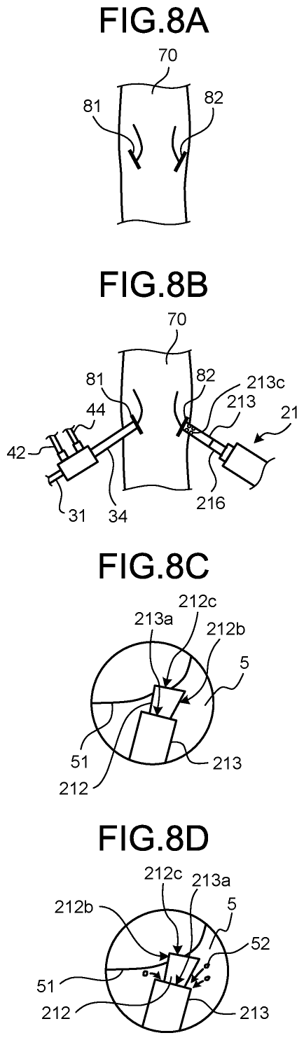

Next, an example of a procedure of an arthroscopic surgery method, which is an endoscopic surgery method using the endoscopic surgery system 1 according to the present embodiment, will be explained. First, as illustrated in FIG. 8A, a surgeon incises the skin 70 near a joint, to make a first portal 81 and a second portal 82. Next, as illustrated in FIG. 8B, the surgeon inserts the arthroscope 31 in the first portal 81 through the inside of the cannula 34. Moreover, the surgeon inserts the ultrasound treatment tool 21 in the second portal 82. The arthroscope 31 and the ultrasound treatment tool 21 may be inserted in either portal of the first portal 81 and the second portal 82. Next, as illustrated in FIG. 8C, the surgeon performs positioning of the treating portion 212b of the ultrasound treatment tool 21 with respect to the part to be treated 51 in the articular cavity 5 while viewing a field of view of the arthroscope. Furthermore, at this time, the surgeon confirms that none of the drain holes 213c formed in the outer sheath 213 of the ultrasound treatment tool 21 is present in the field of view of the arthroscope.

Thereafter, as illustrated in FIG. 8D, the surgeon drives the ultrasound treatment tool 21, to cause the probe 212 to make ultrasonic vibration, and thereby performs treatment of the part to be treated 51 by the cutting area 212c of the treating portion 212b. When treatment is performed, treatment is performed while moving the ultrasound treatment tool 21 and the arthroscope 31 such that the drain hole 213c formed in the outer sheath 213 of the ultrasound treatment tool 21 are positioned outside a field of view of arthroscope. Moreover, the fragments 52 of a living tissue generated at this time are sucked from the suction port 213a of the outer sheath 213 as illustrated in FIG. 8D, to be discharged out of the field of view of arthroscope.

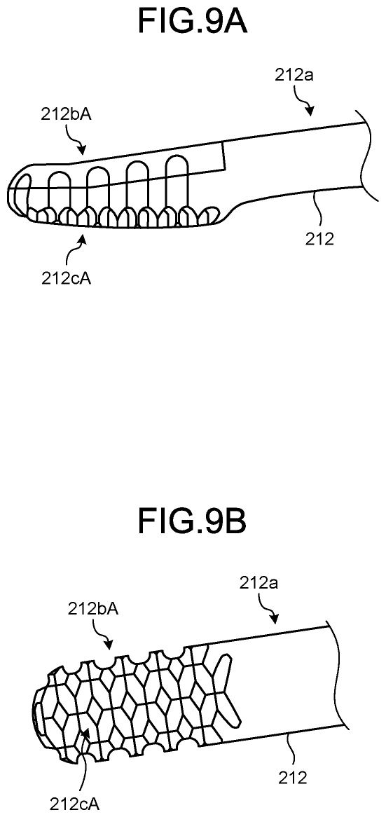

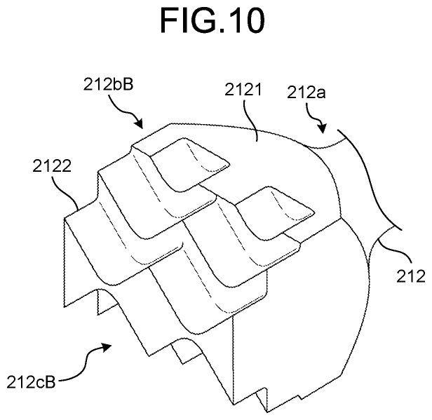

In the ultrasound treatment tool 21 according to the present embodiment, the shape of the treating portion 212b of the probe 212 may be, for example, a shape as illustrated in FIG. 9A or FIG. 9B, or a shape as illustrated in FIG. 10.

A treating portion 212bA of the probe 212 illustrated in FIG. 9A and FIG. 9B has a shape having a cutting area 212cA in a raspatory form with many minute pits and projections. A treating portion 212bB of the probe 212 illustrated in FIG. 10 is constituted of a base portion 2121 and a distal end portion 2122, and has a shape having a cutting area 212cB in a ridge shape in the distal end portion 2122.

In the ultrasound treatment tool 21 according to the present embodiment, the shape, arrangement, number, and the like of the drain holes 213c formed in the outer sheath 213 are not particularly limited, as long as the perfusion solution including the fragments 52 of a living tissue can be drained from the inside of the outer sheath 213.

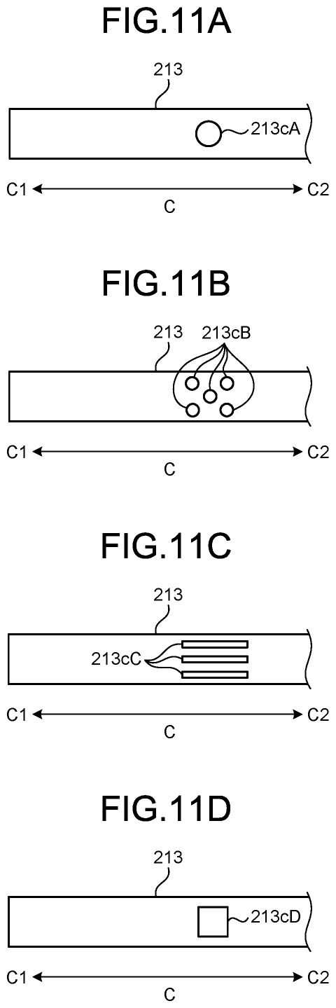

For example, as illustrated in FIG. 11A, a total of four drain holes 213cA in a circular shape may be arranged in a circumferential direction of the outer sheath 213, arranging one each, for example, every 45 degrees. Alternatively, as illustrated in FIG. 11B, plural circular drain holes 213cB may be arranged throughout the circumferential direction of the outer sheath 213 in a staggered alignment. Alternatively, as illustrated in FIG. 11C, plural drain holes 213cC in a rectangular shape lengthy in the longitudinal direction C may be formed throughout the circumferential direction of the outer sheath 213. Alternatively, as illustrated in FIG. 11D, a total of four square drain holes 213cD may be formed in the circumferential direction of the outer sheath 213, arranging one each, for example, every 45 degrees.

Moreover, in the ultrasound treatment tool 21 according to the present embodiment, the inner sheath 214 is not necessarily provided. In this case, space between the outer circumferential surface of the probe main body 212a and the inner circumferential surface of the outer sheath 213 is used as the suction channel 213b, and the perfusion solution sucked from the suction port 213a of the outer sheath 213 passes through the suction channel 213b to be drained out from the drain holes 213c of the outer sheath 213.

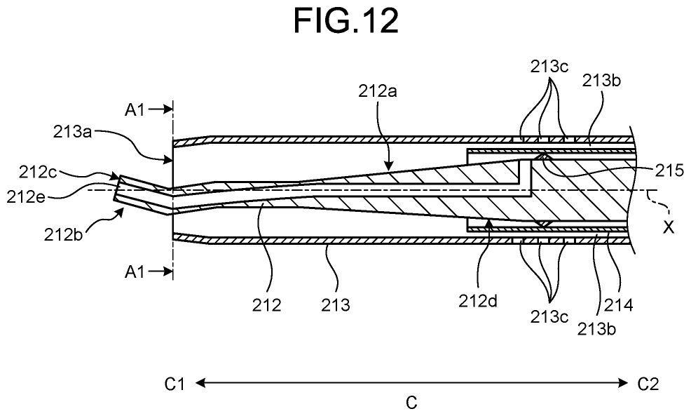

Furthermore, as illustrated in FIG. 12, in the ultrasound treatment tool 21 according to the present embodiment, a through hole 212e that communicates between the cutting area 212c (distal end surface) of the treating portion 212b and the outer circumferential surface of the probe main body 212a may be formed in the probe 212. Thus, the fragments 52 of a living tissue generated near the part to be treated 51 can be discharged through the through hole 212e of the probe 212. Although an opening position of the through hole 212e on the outer circumferential surface of the probe main body 212a is near the drain holes 213c of the outer sheath 213 as illustrated in FIG. 12, but it is not limited thereto. For example, the opening position of the through hole 212e on the outer circumferential surface of the probe main body 212a may be at a portion not covered with the inner sheath 214 in the tapered portion 212d.

Hereinafter, another exemplary embodiment of an ultrasound treatment tool, an ultrasound treatment system, an endoscopic surgery system, and an endoscopic surgery method according to the disclosure will be explained. Because the endoscopic surgery system according to this embodiment is the same as the endoscopic surgery system according to the above embodiment except the configuration of the ultrasound treatment tool, explanation of parts in common with the above embodiment will be omitted as appropriate.

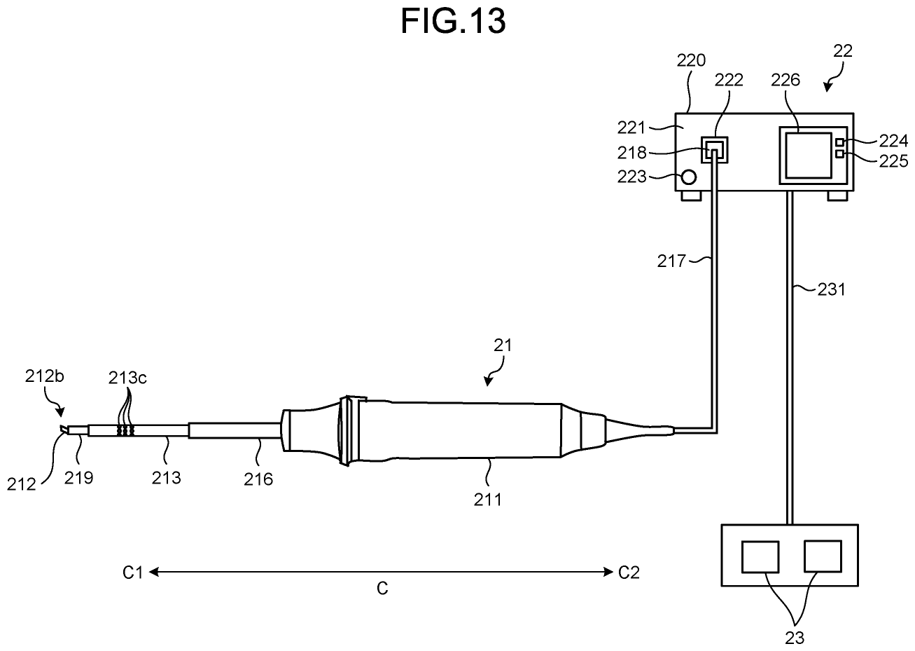

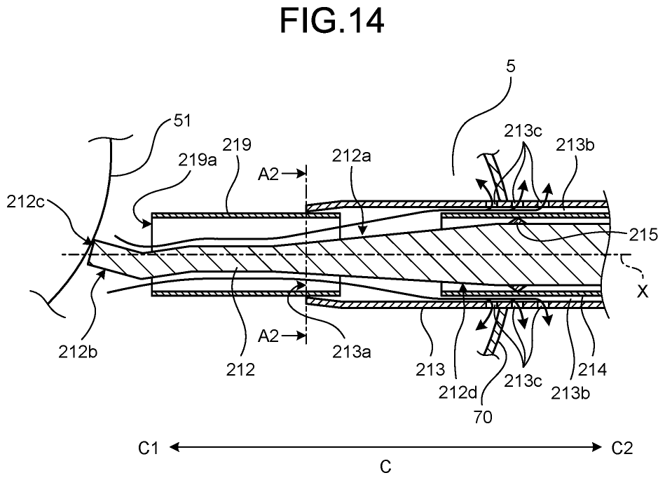

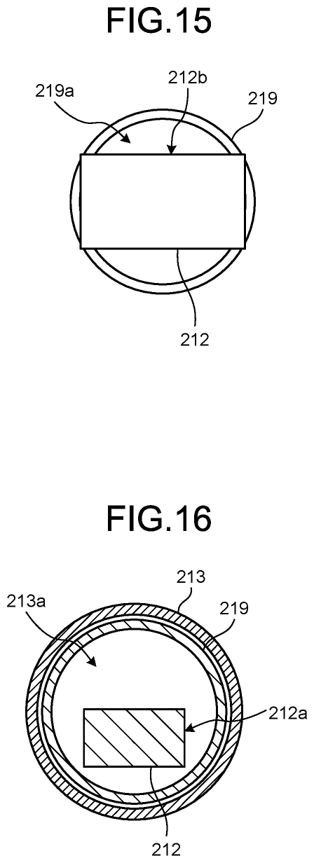

FIG. 13 is a diagram illustrating an entire configuration of the ultrasound treatment system 2 that is included in the endoscopic surgery system 1 according to the present embodiment. FIG. 14 is a diagram illustrating a flow of the perfusion solution that is caused by causing a probe 212 to make ultrasonic vibrations. FIG. 15 is a diagram of the probe 212 and a probe cover 219 viewed along a longitudinal direction from the distal end direction C1 side of the probe 212 to the proximal end direction C2 side.

The ultrasound treatment tool 21 included in the ultrasound treatment system 2 according to the present embodiment includes the rod-shaped probe 212, the outer sheath 213 that covers a circumference of the probe 212 to protect the probe 212, the inner sheath 214 that is arranged inside the outer sheath 213, the seal ring 215, the resin tube 216, the cable 217, the connector 218., and the probe cover 219 that is a third tubular portion. The probe cover 219 is provided to prevent a contact between the probe 212 and the arthroscope 31.

As illustrated in FIG. 14, the probe cover 219 is made from resin, and has a tubular shape thinner than the outer sheath 213, and covers a part of an outer circumferential surface of the probe main body 212a of the probe 212 on the distal end direction cl side. The outer circumferential surface of the probe cover 219 on the proximal end direction C2 side is covered with the outer sheath 213, and a hollow interior of the probe cover 219 and a hollow interior of the outer sheath 213 communicate each other.

At the distal end in the longitudinal direction C of the probe cover 219, a suction port 219a, which is an opening, is formed. Moreover, space is formed between an inner circumferential surface of the probe cover 219 and an outer circumferential surface of the probe main body 212a, and the probe cover 219 is movable relative to the probe main body 212a. As illustrated in FIG. 15, parts of the treating portion 212b of the probe 212, specifically, four corner portions of the treating portion 212b in a rectangular shape protrude to an outer circumferential side relative to the inner circumferential surface of the probe cover 219 when the probe 212 and the probe cover 219 are viewed from the distal end direction C1 side of the probe 212 toward the proximal end direction C2 side along the longitudinal direction. Thus, the proximal end of the treating portion 212b and the distal end of the probe cover 219 come into contact with each other, and movement of the probe cover 219 toward the distal end direction C1 side is thereby restricted by the treating portion 212b, and it is possible to prevent the probe cover 219 from falling off from the probe 212.

In the ultrasound treatment tool 21 according to the present embodiment, by covering a part of the outer circumferential surface of the probe main body 212a of the probe 212 on the distal end direction C1 side with the probe cover 219 that is thinner than the outer sheath 213, the diameter becomes small on the distal end direction C1 side of the probe main body 212a, thereby increasing the suction force. Furthermore, the ultrasound treatment tool 21 according to the present embodiment can improve the operability in treatment of the part to be treated in the narrow articular cavity 5.

In the ultrasound treatment tool 21 according to the present embodiment, as illustrated in FIG. 14, as the probe 212 makes ultrasound vibration in the longitudinal direction C, the perfusion solution including the fragments 52 of a living tissue that are generated in the cutting area 212c flows into the probe cover 219 from the suction port 219a of the probe cover 219, and flows inside the probe cover 219 toward the proximal end direction C2 side along the probe main body 212a. The perfusion solution including the fragments 52 of a living tissue flows into the outer sheath 213, flows inside the outer sheath 213 toward the proximal end direction C2 side, passes through the suction channel 213b, and is drained out from the drain holes 213c of the outer sheath 213. Thus, the fragments 52 of a living tissue can be sucked to be removed near a portion at which the fragments 52 of a living tissue are generated. Therefore, because the fragments 52 of a living tissue can be removed before the fragments 52 of a living tissue are scattered around, the fragments 52 of a living tissue can be removed efficiently, and turbidity of the perfusion solution can be reduced, to obtain a clear visibility in a field of view of an arthroscope.

Moreover, in the ultrasound treatment tool 21 according to the present embodiment, the total sum of areas of the drain holes 213c that are formed in the outer sheath 213 is larger than the opening area of the suction port 213a of the outer sheath 213 on an A2-A2 cross-section illustrated in FIG. 16. This enables to make the amount of perfusion solution drained from the drain holes 213c larger than the amount of perfusion solution sucked through the suction port 213a of the outer sheath 213, and the perfusion solution can be efficiently drained from the inside of the outer sheath 213.

The opening area of the suction port 213a in the ultrasound treatment tool 21 according to the present embodiment is, as illustrated in FIG. 16, an area acquired by subtracting a cross-sectional area of the probe 212 and a cross-sectional area of the probe cover 219 from a cross-sectional area of the suction port 213a (area of a region surrounded by the inner circumferential surface of the outer sheath 213 on the A2-A2 cross-section) when the A2-A2 cross-section perpendicular to the axis of the probe 212 is viewed from the distal end direction C1 side toward the proximal end direction C2 side along the longitudinal direction C.

Furthermore, by making the inner diameter of the probe cover 219 large, a flow of cavitation in the distal end portion of the probe 212 generated by ultrasonic vibration can be induced to a flow along the probe main body 212a.

The probe cover 219 may be in intimate contact with the probe main body 212a of the probe 212. In this case, as the probe 212 makes ultrasonic vibration in the longitudinal direction C, the perfusion solution including the fragments 52 of a living tissue generated in the cutting area 212c flows inside the outer sheath 213 from the suction port 213a of the outer sheath 213, flows inside the outer sheath 213 toward the proximal end direction C2 side, passes through the suction channel 213b, and drained out from the drain holes 213c of the outer sheath 213.

Hereinafter, a further exemplary embodiment of an ultrasound treatment tool, an ultrasound treatment system, an endoscopic surgery system, and an endoscopic surgery method according to the disclosure will be explained. Because the endoscopic surgery system according to the present embodiment is the same as the endoscopic surgery system according to the above embodiment shown in FIGS. 1-12 except that a suction pump device 6 is provided in the ultrasound treatment system, explanation of parts in common with the above embodiment will be omitted as appropriate.

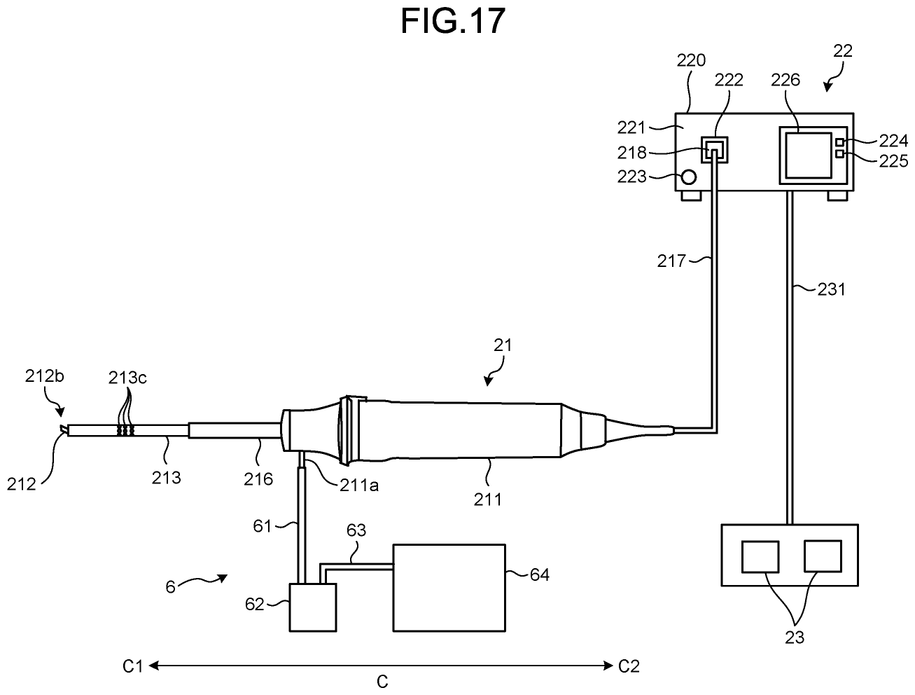

FIG. 17 is a diagram illustrating an entire configuration of the ultrasound treatment system 2 that is included in the endoscopic surgery system 1 according to the present embodiment. The ultrasound treatment system 2 according to the present embodiment further includes the suction pump device 6 in addition to the ultrasound treatment tool 21, the driving device 22, and the foot switch 23.

In the treatment-tool main body 211 of the ultrasound treatment tool 21, a connecting portion 211a that communicates with the suction channel 213b formed between the outer sheath 213 and the inner sheath 214 is provided.

The suction pump device 6 includes a drainage tube 61, a collecting container 62, a suction tube 63, and a suction pump 64. A first end of the drainage tube 61 is connected to the connecting portion 221a of the ultrasound treatment tool 21, and a second end of the drainage tube 61 is connected to the collecting container 62. A first end of the suction tube 63 is connected to the collecting container 62, and a second end of the suction tube 63 is connected to the suction pump 64. The suction pump 64 is constituted of a vacuum pump, or the like.

In the ultrasound treatment system 2 according to the present embodiment, similarly to the ultrasound treatment system 2 according to the above embodiment (FIGS. 1-12), as the probe 212 of the ultrasound treatment tool 21 makes ultrasonic vibration in the longitudinal direction C, the perfusion solution including the fragments 52 of a living tissue generated in the cutting area 212c flows into the outer sheath 213 from the suction port 213a of the outer sheath 213. The perfusion solution flows inside the outer sheath 213 toward the proximal end direction C2 side, passes through the suction channel 213b, and is drained out from drain holes 213c of the outer sheath 213.

Moreover, in the ultrasound treatment system 2 according to the present embodiment, by operating the suction pump 64 of the suction pump device 6, the perfusion solution inside the suction channel 213b is drained to collecting container 62 through the drainage tube 61 from the connecting portion 211a of the treatment-tool main body 211.

In the ultrasound treatment system 2 according to the present embodiment, not just draining the perfusion solution including the fragments 52 of a living tissue from the drain holes 213c of the outer sheath 213 by causing the probe 212 to make ultrasonic vibration, by using the suction pump device 6, the fragments 52 of a living tissue can be sucked to be removed efficiently from a periphery of the part to be treated 51. Therefore, turbidity of the perfusion solution around the part to be treated 51 can be reduced, to obtain clear visibility in a field of view of arthroscope.

Moreover, the ultrasound treatment tool 21 included in the ultrasound treatment system 2 according to the present embodiment may include the probe cover 219 illustrated in FIG. 13 and FIG. 14, similarly to the ultrasound treatment tool 21 according to the above embodiment shown in FIGS. 13-16. In this case, by operating the suction pump 64 of the suction pump device 6, the perfusion solution including the fragments 52 of a living tissue generated in the cutting area 212c flows into the probe cover 219 from the suction port 219a of the probe cover 219, the perfusion solution flows into the outer sheath 212 from the inside of the probe cover 219, and is drained to the collecting container 62 through the drainage tube 61 connected to the connecting portion 211a of the treatment-tool main body 211.

The disclosure can provide an ultrasound treatment tool, an ultrasound treatment system, an endoscopic surgery system, and an endoscopic surgery method that can suppress deterioration of visibility in a field of view of an arthroscope due to turbidity of a perfusion solution.

An ultrasound treatment tool, an ultrasound treatment system, an endoscopic surgery system, and an endoscopic surgery method according to the disclosure produce an effect of suppressing deterioration of visibility in a field of view of an arthroscope due to turbidity of a perfusion solution.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound treatment tool comprising:
a probe that includes a main body and a treating portion, the main body being configured to transmit ultrasonic vibration input from a proximal end side of the main body to a distal end side of the main body, the treating portion being arranged on the distal end side of the main body and being configured to treat a target site by transmitting the ultrasonic vibration in a state in which the target site is in a liquid; and
a first tubular portion that is formed in a tubular shape and includes an inner lumen through which the probe is inserted, the first tubular portion including:
at least one through hole that is formed in a wall of the first tubular portion and extends between an inner circumferential surface and an outer circumferential surface of the first tubular portion so as to be in fluid communication with a liquid channel in the inner lumen of the first tubular portion, and
an opening to the inner lumen that is formed at a distal end of the first tubular portion,
a total opening area of the at least one through hole being larger than a cross-sectional opening area of the liquid channel at the opening.

2. The ultrasound treatment tool according to claim 1, wherein
the liquid channel is formed by a gap between the inner circumferential surface of the first tubular portion at the opening and an outer surface of the probe at the opening, and the cross-sectional opening area of the liquid channel is a cross-sectional area of the gap at the opening.

3. The ultrasound treatment tool according to claim 1, wherein the cross-sectional opening area of the liquid channel is an area acquired by subtracting a cross-sectional area of the probe at the opening from a cross-sectional area of the inner lumen of the first tubular portion at the opening.

4. The ultrasound treatment tool according to claim 1, wherein the probe extends through the opening to the inner lumen of the first tubular portion.

5. The ultrasound treatment tool according to claim 1, wherein the first tubular portion includes two or more through holes, and the total opening area is a sum of opening areas of the two or more through holes.

6. The ultrasound treatment tool according to claim 1, further comprising
a third tubular portion that is formed in a tubular shape to cover the distal end side of the main body of the probe, wherein
the third tubular portion is covered by the first tubular portion on a proximal end side, and
the cross-sectional opening area of the liquid channel at the opening is larger than a cross-sectional opening area of a space between the inner circumferential surface of the first tubular portion at the opening and an outer circumferential surface of the third tubular portion at the opening.

7. The ultrasound treatment tool according to claim 1, further comprising
a second tubular portion that is provided between the main body of the probe and the first tubular portion, wherein
the at least one through hole is configured to communicate with space formed between the inner circumferential surface of the first tubular portion and an outer circumferential surface of the second tubular portion.

8. An ultrasound treatment tool comprising:
a probe that includes a main body and a treating portion, the main body extending along a central axis from a proximal end side to a distal end side and being configured to transmit ultrasonic vibration input from the proximal end side to the distal end side of the main body along the central axis, the treating portion being arranged on the distal end side of the main body and being configured to treat a target site with the transmitted ultrasonic vibration in a state in which the target site is in a liquid; and
a first tubular portion that is formed in a tubular shape with the probe being inserted therein, the first tubular portion including:
at least one through hole that is formed in a wall of the first tubular portion and extends between an inner circumferential surface and an outer circumferential surface of the first tubular portion so as to be in fluid communication with a liquid channel of the first tubular portion, and
an opening that is formed at a distal end of the first tubular portion,
an antinode position of a standing wave of the ultrasonic vibration being positioned at the treating portion, and
the at least one through hole being formed at a position along the central axis that corresponds to a node position nearest to the antinode position, or formed on a distal end side relative to the node position nearest to the antinode position.

9. The ultrasound treatment tool according to claim 1, wherein
an outer circumferential surface of the main body in the probe includes a tapered portion in which a thickness of the main body decreases along a direction toward the distal end side from the proximal end side, and
the at least one through hole is formed in the first tubular portion at a position facing the tapered portion.

10. An ultrasound treatment system composing:
the ultrasound treatment tool according to claim 1; and
a driving device configured to drive the ultrasound treatment tool.

11. An endoscopic surgery system comprising:
the ultrasound treatment system according to claim 10;
an endoscope configured to acquire image data of the target site in an articular cavity; and
a perfusion device configured to perfuse the liquid into the articular cavity.

12. The ultrasound treatment tool according to claim 8, wherein the antinode position is positioned at a distal end of the treating portion.

13. The ultrasound treatment tool according to claim 8, wherein the treating portion is configured to contact the target site to cut living tissue at the target site, thereby generating fragments of the living tissue in the liquid, and the ultrasound treatment tool is configured to suck the liquid including the fragments of the living tissue through the opening in the first tubular portion such that the liquid flows through the liquid channel in a proximal direction toward the node position and is drained from the at least one-through hole.

14. The ultrasound treatment tool according to claim 8, wherein:
an outer surface of the main body in the probe includes a tapered portion in which a thickness of the main body decreases along the central axis toward the distal end side from the proximal end side, and
the at least one through hole is formed on the distal end side relative to the node position at a position facing the tapered portion.

15. The ultrasound treatment tool according to claim 14, wherein the at least one through hole faces a proximal end portion of the tapered portion.

16. The ultrasound treatment tool according to claim 8, wherein:
an outer surface of the main body in the probe includes a tapered portion in which a thickness of the main body decreases along the central axis toward the distal end side from the proximal end side, and the at least one through hole includes a first through hole formed on the distal end side relative to the node position at a position facing the tapered portion.

17. The ultrasound treatment tool according to claim 16, wherein the at least one through hole further includes a second through hole formed at the position along the central axis corresponding to the node position.
